(19) United States
(12) Patent Application Publication
(10) Pub. No.: US 2010/0179203 A1
(43) Pub. Date: Jul. 15, 2010

(54) NOVEL PYRAZOLE DERIVATIVES USEFUL AS POTASSIUM CHANNEL MODULATORS

(76) Inventors: Antonio Nardi, Ballerup (DK); Jeppe Kejser Christensen, København (DK); David Spencer Jones, Smørum (DK)

Correspondence Address: BIRCH STEWART KOLASCH & BIRCH, PO BOX 747, FALLS CHURCH, VA, 22040-0747, US

(21) Appl. No.: 12/667,474

(22) PCT Filed: Jun. 27, 2008
(86) PCT No.: PCT/EP08/58221

§ 371 Date: Dec. 31, 2009

Related U.S. Application Data
(60) Provisional application No. 60/948,322, filed on Jul. 6, 2007.

(30) Foreign Application Priority Data
Jul. 4, 2007 (DK) PA 2007 00988

(51) Int. Cl. A61K 31/41; C07D 231/10; A61K 31/415; C07D 257/04; A61P 1/00; A61P 9/00; A61P 11/00; A61P 13/00; A61P 25/00; A61P 27/00; A61P 29/00

(52) U.S. Cl. 514/381; 548/375.1; 514/406; 548/373.1; 548/250

(57) ABSTRACT

This invention relates to novel pyrazole derivatives that are found to be potent modulators of potassium channels and, as such, are valuable candidates for the treatment of diseases or disorders as diverse as those which are responsive to the modulation of potassium channels.

### TECHNICAL FIELD

This invention relates to novel pyrazole derivatives that are found to be potent modulators of potassium channels and, as such, are valuable candidates for the treatment of diseases or disorders as diverse as those which are responsive to the modulation of potassium channels.

### BACKGROUND ART

Ion channels are cellular proteins that regulate the flow of ions through cellular membranes of all cells and are classified by their selective permeability to the different of ions (potassium, chloride, sodium etc.). Potassium channels, which represent the largest and most diverse sub-group of ion channels, selectively pass potassium ions and, doing so, they principally regulate the resting membrane potential of the cell and/or modulate their level of excitation.

Dysfunction of potassium channels, as well as other ion channels, generates loss of cellular control resulting in altered physiological functioning and disease conditions. Ion channel blockers and openers, by their ability to modulate ion channel function and/or regain ion channel activity in acquired or inherited channelopathies, are being used in the pharmacological treatment of a wide range of pathological diseases and have the potential to address an even wider variety of therapeutic indications. For instance, the primary indications for potassium channel openers encompass conditions as diverse as diabetes, arterial hypertension, cardiovascular diseases, urinary incontinence, atrial fibrillation, epilepsy, pain, and cancer.

Among the large number of potassium channel types, the large-conductance calcium-activated potassium channel subtype is an obvious site for pharmacological intervention and for the development of new potassium channel modulators. Their physiological role has been especially studied in the nervous system, where they are key regulators of neuronal excitability and of neurotransmitter release, and in smooth muscle, where they are crucial in modulating the tone of vascular, broncho-tracheal, urethral, uterine or gastro-intestinal musculature.

Given these implications, small agents with BK-opening properties could have a potentially powerful influence in the modulation and control of numerous consequences of muscular and neuronal hyperexcitability, such as asthma, urinary incontinence and bladder spasm, gastroenteric hypermotility, psychoses, post-stroke neuroprotection, convulsions, epilepsy, anxiety and pain. As far as the cardiovascular system is concerned, the physiological function of these ion channels represents a fundamental steady state mechanism, modulating vessel depolarisation, vasoconstriction and increases of intravascular pressure, and the development of selective activators of BK channels is seen as a potential pharmacotherapy of vascular diseases, including hypertension, erectile dysfunction, coronary diseases and vascular complications associated with diabetes or hypercholesterolemia.

GB 1373212 and Bratenko et al.; Russian Journal of Organic Chemistry 2002 38 (8) 1171-1177, describe 1,3-diarylpyrazolyl carboxylic acids useful as anti-inflammatory agents.

### SUMMARY OF THE INVENTION

It is an object of the invention to provide novel pyrazole derivatives useful as potassium channel modulators. The pyrazole derivatives of the invention may be characterised by Formula I

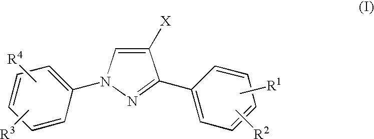

an isomer thereof or a mixture of its isomers, or an N-oxide thereof, or a pharmaceutically acceptable salt thereof, wherein

X represents

a tetrazolyl-alkyl group, an oxadiazolonyl-alkyl group, an [(N-alkyl-sulfonyl)carbamoyl]-alkyl group, 2-cyano-acrylic acid, 2-cyano-acryloyl-alkylsulfonamide or 2-cyano-acryloyl-phenylsulfonamide;

or a group of formula CH═CH—W or CH₂—CH₂—W, wherein W represents a tetrazolyl group, N-alkylsulfonylcarboxamide, carboxy, N-cyanocarboxamide or 5-oxo-4,5-dihydro-[1,2,4]oxadiazol-3-yl;

R¹ and R², independently of each other, represent hydrogen, halo, hydroxy or phenyl, which phenyl may optionally be optionally substituted one or more times with halo; and

R³ and R⁴, independently of each other, represent hydrogen, halo, trifluoromethyl, hydroxy, alkoxy or phenyl.

In another aspect the invention provides pharmaceutical compositions comprising a therapeutically effective amount of a pyrazole derivative of the invention.

In a third aspect the invention relates to the use of the pyrazole derivatives of the invention for the manufacture of pharmaceutical compositions.

In a further aspect the invention provides a method of treatment, prevention or alleviation of a disease or a disorder or a condition of a living animal body, including a human, which disorder, disease or condition is responsive to modulation of potassium channels, which method comprises the step of administering to such a living animal body in need thereof, a therapeutically effective amount of the pyrazole derivative of the invention.

Other objects of the invention will be apparent to the person skilled in the art from the following detailed description and examples.

### DETAILED DISCLOSURE OF THE INVENTION

In its first aspect the invention provides novel pyrazole derivatives of Formula I

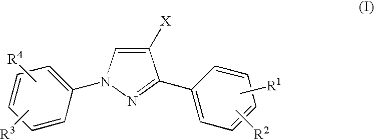

an isomer thereof or a mixture of its isomers, or an N-oxide thereof, or a pharmaceutically acceptable salt thereof, wherein

X represents a tetrazolyl-alkyl group, an oxadiazolonyl-alkyl group, an [(N alkyl-sulfonyl)carbamoyl]-alkyl group, 2-cyano-acrylic acid, 2-cyano-acryloyl-alkylsulfonamide or 2-cyano-acryloyl-phenylsulfonamide; or a group of formula CH═CH—W or CH₂—CH₂—W, wherein W represents a tetrazolyl group, N-alkylsulfonylcarboxamide, carboxy, N-cyanocarboxamide or 5-oxo-4,5-dihydro-[1,2,4]oxadiazol-3-yl;

R¹ and R², independently of each other, represent hydrogen, halo, hydroxy or phenyl, which phenyl may optionally be optionally substituted one or more times with halo; and

R³ and R⁴, independently of each other, represent hydrogen, halo, trifluoromethyl, hydroxy, alkoxy or phenyl.

In a preferred embodiment the pyrazole derivative of the invention is a compound of Formula IA

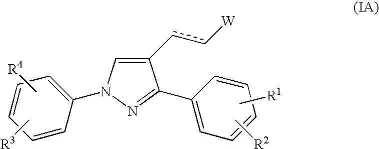

an isomer thereof or a mixture of its isomers, or a pharmaceutically acceptable salt thereof, wherein

[symbol] designates an optional double bond (i.e. a single or a double bond);

W represents 1H-tetrazol-5-yl, N-methylsulfonylcarboxamide, carboxy, N-cyanocarboxamide or 5-oxo-4,5-dihydro-[1,2,4]oxadiazol-3-yl;

R¹ and R², independently of each other, represent hydrogen, halo, hydroxyl, or phenyl, which phenyl may optionally be optionally substituted one or more times with halo; and

R³ and R⁴, independently of each other, represent hydrogen, halo, trifluoromethyl, hydroxy or phenyl.

In a more preferred embodiment the pyrazole derivative of the invention is a compound of Formula IA, wherein [symbol] designates a single (covalent) bond or a double bond.

In another more preferred embodiment the pyrazole derivative of the invention is a compound of Formula IA, wherein [symbol] designates a single (covalent) bond.

In a third more preferred embodiment the pyrazole derivative of the invention is a compound of Formula IA, wherein [symbol] designates a double bond.

In another preferred embodiment the pyrazole derivative of the invention is a compound of Formula I, or a pharmaceutically acceptable salt thereof, wherein X represents a tetrazolyl-alkyl group, an oxadiazolonyl-alkyl group, an [(N-alkyl-sulfonyl)carbamoyl]-alkyl group, 2-cyano-acrylic acid, 2-cyano-acryloyl-alkylsulfonamide or 2-cyano-acryloyl-phenylsulfonamide.

In a more preferred embodiment X represents a tetrazolyl-alkyl group, and in particular 1H-tetrazol-5-yl-methyl.

In another more preferred embodiment X represents an oxadiazolonyl-alkyl group, and in particular 4H-[1,2,4]oxadiazol-5-one-methyl.

In a third more preferred embodiment X represents an [(N-alkyl-sulfonyl)carbamoyl]-alkyl group, and in particular N-methylsulfonylacetamide.

In a fourth more preferred embodiment X represents 2-cyano-acrylic acid (i.e. CH═CH(CN)—COOH).

In a fifth more preferred embodiment X represents 2-cyano-acryloyl-alkylsulfonamide (i.e. CH═CH(CN)—CO—NH—SO₂-alkyl), and in particular 2-cyano-acryloyl-methylsulfonamide.

In a sixth more preferred embodiment X represents 2-cyano-acryloyl-phenylsulfonamide (i.e. CH═CH(CN)—CO—NH—SO₂-phenyl).

In another preferred embodiment the pyrazole derivative of the invention is a compound of Formula i, or a pharmaceutically acceptable salt thereof, wherein X represents a group of formula CH═CH—W or CH₂—CH₂—W, wherein W represents a tetrazolyl group, N-alkylsulfonylcarboxamide, carboxy, N-cyanocarboxamide or 5-oxo-4,5-dihydro-[1,2,4]oxadiazol-3-yl.

In a more preferred embodiment the pyrazole derivative of the invention is a compound of Formula I or IA, or a pharmaceutically acceptable salt thereof, wherein W represents 1H-tetrazol-5-yl, N-methylsulfonylcarboxamide, carboxy, N-cyanocarboxamide or 5-oxo-4,5-dihydro-[1,2,4]oxadiazol-3-yl.

In another more preferred embodiment the pyrazole derivative of the invention is a compound of Formula I or IA, wherein W represents a tetrazolyl group.

In a third more preferred embodiment the pyrazole derivative of the invention is a compound of Formula I or IA, wherein W represents 1H-tetrazol-5-yl.

In a fourth more preferred embodiment the pyrazole derivative of the invention is a compound of Formula I or IA, wherein W represents N-alkylsulfonylcarboxamide.

In a fifth more preferred embodiment the pyrazole derivative of the invention is a compound of Formula I or IA, wherein W represents N-methylsulfonylcarboxamide.

In a sixth more preferred embodiment the pyrazole derivative of the invention is a compound of Formula I or IA, wherein W represents carboxy.

In a seventh more preferred embodiment the pyrazole derivative of the invention is a compound of Formula I or IA, wherein W represents N-cyanocarboxamide.

In an eighth more preferred embodiment the pyrazole derivative of the invention is a compound of Formula I or IA, wherein W represents 5-oxo-4,5-dihydro-[1,2,4]oxadiazol-3-yl.

In a third preferred embodiment the pyrazole derivative of the invention is a compound of Formula I or IA, or a pharmaceutically acceptable salt thereof, wherein R¹ and R², independently of each other, represent hydrogen, halo, hydroxy or phenyl, which phenyl may optionally be optionally substituted one or more times with halo.

In a more preferred embodiment the pyrazole derivative of the invention is a compound of Formula I or IA, wherein R¹ and R², independently of each other, represent hydrogen, halo, hydroxy or phenyl.

In another more preferred embodiment the pyrazole derivative of the invention is a compound of Formula I or IA, wherein R¹ and R², independently of each other, represent hydrogen or halo, and in particular fluoro or chloro.

In a third more preferred embodiment the pyrazole derivative of the invention is a compound of Formula I or IA, wherein one of R¹ and R² represents hydrogen; and the other of R¹ and R² represents halo, hydroxy or phenyl, which phenyl may optionally be optionally substituted one or more times with halo.

In a fourth more preferred embodiment the pyrazole derivative of the invention is a compound of Formula I or IA, wherein one of R¹ and R² represents hydrogen; and the other of R¹ and R² represents halo, hydroxy or phenyl.

In a fifth more preferred embodiment the pyrazole derivative of the invention is a compound of Formula I or IA, wherein one of R¹ and R² represents hydrogen; and the other of R¹ and R² represents halo, and in particular chloro.

In a sixth more preferred embodiment the pyrazole derivative of the invention is a compound of Formula I or IA, wherein one of R¹ and R² represents halo, and in particular fluoro; and the other of R¹ and R² represents halo, and in particular chloro.

In a fourth preferred embodiment the pyrazole derivative of the invention is a compound of Formula I or IA, or a pharmaceutically acceptable salt thereof, wherein R³ and R⁴, independently of each other, represent hydrogen, halo, trifluoromethyl, hydroxy, alkoxy or phenyl.

In a more preferred embodiment the pyrazole derivative of the invention is a compound of Formula I or IA, wherein R³ and R⁴, independently of each other, represent hydrogen, halo, trifluoromethyl or alkoxy.

In another more preferred embodiment the pyrazole derivative of the invention is a compound of Formula I or IA, wherein R³ and R⁴, independently of each other, represent hydrogen, halo or trifluoromethyl.

In a third more preferred embodiment the pyrazole derivative of the invention is a compound of Formula I or IA, wherein one of R³ and R⁴ represents hydrogen; and the other of R³ and R⁴ represents halo, trifluoromethyl, hydroxy, alkoxy or phenyl.

In a fourth more preferred embodiment the pyrazole derivative of the invention is a compound of Formula I or IA, wherein one of R³ and R⁴ represents hydrogen; and the other of R³ and R⁴ represents halo, trifluoromethyl, hydroxy or phenyl.

In a fifth more preferred embodiment the pyrazole derivative of the invention is a compound of Formula I or IA, wherein one of R³ and R⁴ represents hydrogen; and the other of R³ and R⁴ represents halo, and in particular bromo.

In a sixth more preferred embodiment the pyrazole derivative of the invention is a compound of Formula I or IA, wherein both of R³ and R⁴, independently of each other, represent halo and/or trifluoromethyl.

In a seventh more preferred embodiment the pyrazole derivative of the invention is a compound of Formula I or IA, wherein both of R³ and R⁴ represent halo.

In an eighth more preferred embodiment the pyrazole derivative of the invention is a compound of Formula i or IA, wherein both of R³ and R⁴ represent trifluoromethyl.

In a ninth more preferred embodiment the pyrazole derivative of the invention is a compound of Formula I or IA, wherein one of R³ and R⁴ represents halo, and in particular chloro; and the other of R³ and R⁴ represents alkoxy, and in particular methoxy.

In a most preferred embodiment the pyrazole derivative of the invention is
(E)-3-[1-(3,5-Bis-trifluoromethyl-phenyl)-3-(3-chloro-phenyl)-1H-pyrazol-4-yl]-acrylic acid;
(E)-3-[1-(3,5-Bis-trifluoromethyl-phenyl)-3-(3-chloro-phenyl)-1H-pyrazol-4-yl]-N-cyanoacrylamide;
(E)-3-[3-(4-Chloro-3-fluoro-phenyl)-1-(3-chloro-4-methoxy-phenyl)-1H-pyrazol-4-yl]-acrylic acid;
5-{(E)-2-[1-(3,5-Bis-trifluoromethyl-phenyl)-3-(3-chloro-phenyl)-1H-pyrazol-4-yl]-vinyl}-1H-tetrazole;
5-{(E)-2-[3-(4-Chloro-3-fluoro-phenyl)-1-(3-chloro-4-methoxy-phenyl)-1H-pyrazol-4-yl]-vinyl}-1H-tetrazole;
N-{(E)-3-[3-(4-Chloro-3-fluoro-phenyl)-1-(3-chloro-4-methoxy-phenyl)-1H-pyrazol-4-yl]acryloyl}-methanesulfonamide;
3-[1-(4-Bromo-phenyl)-3-(4-chloro-phenyl)-1H-pyrazol-4-yl]-N-cyanopropionamide;
N-{3-[1-(4-Bromo-phenyl)-3-(4-chloro-phenyl)-1H-pyrazol-4-yl]-propionyl}-methanesulfonamide;
5-{2-[1-(4-Bromo-phenyl)-3-(4-chloro-phenyl)-1H-pyrazol-4-yl]-ethyl}-1H-tetrazole;
3-[3-(4-Chloro-3-fluoro-phenyl)-1-(3-chloro-4-methoxy-phenyl)-1H-pyrazol-4-yl]-propionic acid;
N-{3-[3-(4-Chloro-3-fluoro-phenyl)-1-(3-chloro-4-methoxy-phenyl)-1H-pyrazol-4-yl]-propionyl}-methanesulfonamide;
(E)-3-[1-(3,5-Bis-trifluoromethyl-phenyl)-3-(3-chloro-phenyl)-1H-pyrazol-4-yl]-2-cyano-acrylic acid;
N-{(E)-3-[1-(3,5-Bis-trifluoromethyl-phenyl)-3-(3-chloro-phenyl)-1H-pyrazol-4-yl]-2-cyano-acryloyl}-methanesulfonamide;
(E)-3-[3-(4-Chloro-3-fluoro-phenyl)-1-(3-chloro-4-methoxy-phenyl)-1H-pyrazol-4-yl]-2-cyano-acrylic acid;
N-{(E)-3-[3-(4-Chloro-3-fluoro-phenyl)-1-(3-chloro-4-methoxy-phenyl)-1H-pyrazol-4-yl]-2-cyano-acryloyl}-benzenesulfonamide;
N-{(E)-3-[3-(4-Chloro-3-fluoro-phenyl)-1-(3-chloro-4-methoxy-phenyl)-1H-pyrazol-4-yl]-2-cyano-acryloyl}-methanesulfonamide;
3-[1-(3,5-Bis-trifluoromethyl-phenyl)-3-(3-chloro-phenyl)-1H-pyrazol-4-ylmethyl]-4H-[1,2,4]oxadiazol-5-one;
N-{2-[3-(4-Chloro-3-fluoro-phenyl)-1-(3-chloro-4-methoxy-phenyl)-1H-pyrazol-4-yl]-acetyl}-methanesulfonamide; or
5-[3-(4-Chloro-3-fluoro-phenyl)-1-(3-chloro-4-methoxy-phenyl)-1H-pyrazol-4-ylmethyl]-1H-tetrazole;

or an isomer thereof or a mixture of its isomers, or an N-oxide thereof, or a pharmaceutically acceptable salt thereof.

Any combination of two or more of the embodiments described herein is considered within the scope of the present invention.

### Definition of Substituents

In the context of this invention halo represents fluoro, chloro, bromo or iodo.

### Pharmaceutically Acceptable Salts

The pyrazole derivatives of the invention may be provided in any form suitable for the intended administration. Suitable forms include pharmaceutically (i.e. physiologically) acceptable salts, and pre- or prodrug forms of the pyrazole derivative of the invention.

Examples of pharmaceutically acceptable addition salts include, without limitation, the non-toxic inorganic and organic acid addition salts such as the hydrochloride, the hydrobromide, the nitrate, the perchlorate, the phosphate, the sulphate, the formate, the acetate, the aconate, the ascorbate, the benzenesulphonate, the benzoate, the cinnamate, the citrate, the embonate, the enantate, the fumarate, the glutamate, the glycolate, the lactate, the maleate, the malonate, the mandelate, the methanesulphonate, the naphthalene-2-sulphonate derived, the phthalate, the salicylate, the sorbate, the stearate, the succinate, the tartrate, the toluene-p-sulphonate, and the like. Such salts may be formed by procedures well known and described in the art.

Examples of pharmaceutically acceptable cationic salts of a pyrazole derivative of the invention include, without limitation, the sodium, the potassium, the calcium, the magnesium, the lithium, and the ammonium salt, and the like, of a pyrazole derivative of the invention containing an anionic group. Such cationic salts may be formed by procedures well known and described in the art.

### Steric Isomers

It will be appreciated by those skilled in the art that the compounds of the present invention may exist in different stereoisomeric forms, including enantiomers, diastereorners, as well as geometric isomers (cis-trans isomers). The invention includes all such isomers and any mixtures thereof including racemic mixtures.

Racemic forms can be resolved into the optical antipodes by known methods and techniques. One way of resolving racemates into the optical antipodes is based upon chromatography on an optical active matrix. Racemic compounds of the present invention can thus be resolved into their optical antipodes, e.g., by fractional crystallisation of D- or L-(tartrates, mandelates, or camphorsulphonate) salts for example.

Additional methods for the resolving the optical isomers are known in the art. Such methods include those described by Jaques J, Collet A, & Wilen S in “Enantiomers, Racemates, and Resolutions”, John Wiley and Sons, New York (1981).

Optical active compounds can also be prepared from optically active starting materials or intermediates.

### Methods of Preparation

The compounds according to the invention may be prepared by conventional methods for chemical synthesis, e.g. those described in the working examples.

### Biological Activity

The pyrazole derivatives of the invention have been found to possess potassium channel modulating activity as measured by standard electrophysiological methods. Due to their activity at the potassium channels, the pyrazole derivatives of the invention are considered useful for the treatment of a wide range of diseases and conditions.

In a special embodiment, the pyrazole derivatives of the invention are considered useful for the treatment, prevention or alleviation of a respiratory disease, epilepsy, partial epilepsy, convulsions, seizures, absence seizures, vascular spasms, coronary artery spasms, motor neuron diseases, myokymia, renal disorders, polycystic kidney disease, bladder hyperexcitability, bladder spasms, urinogenital disorders, urinary incontinence, bladder outflow obstruction, erectile dysfunction, gastrointestinal dysfunction, gastrointestinal hypomotility disorders, gastrointestinal motility insufficiency, postoperative ileus, constipation, gastroesophageal reflux disorder, secretory diarrhoea, an obstructive or inflammatory airway disease, ischaemia, cerebral ischaemia, ischaemic heart disease, angina pectoris, coronary heart disease, ataxia, traumatic brain injury, stroke, Parkinson's disease, bipolar disorder, psychosis, schizophrenia, autism, anxiety, mood disorders, depression, manic depression, psychotic disorders, dementia, learning deficiencies, age related memory loss, memory and attention deficits, Alzheimer's disease, amyotrophic lateral sclerosis (ALS), dysmenorrhoea, narcolepsy, sleeping disorders, sleep apnoea, Reynaud's disease, intermittent claudication, Sjogren's syndrome, xerostomia, cardiovascular disorders, hypertension, myotonic dystrophy, myotonic muscle dystrophia, spasticity, xerostomia, diabetes Type II, hyperinsulinemia, premature labour, cancer, brain tumours, inflammatory bowel disease, irritable bowel syndrome, colitis, colitis Crohn, immune suppression, hearing loss, migraine, pain, neuropathic pain, inflammatory pain, trigeminal neuralgia, vision loss, rhinorrhoea, ocular hypertension (glaucoma) or baldness.

In a more preferred embodiment, the pyrazole derivatives of the invention are considered useful for the treatment, prevention or alleviation of a respiratory disease, urinary incontinence, erectile dysfunction, anxiety, epilepsy, psychosis, schizophrenia, bipolar disorder, depression, amyotrophic lateral sclerosis (ALS), Parkinson's disease or pain.

In another more preferred embodiment, the pyrazole derivatives of the invention are considered useful for the treatment, prevention or alleviation of psychosis, schizophrenia, bipolar disorder, depression, epilepsy, Parkinson's disease or pain.

In a third more preferred embodiment, the pyrazole derivatives of the invention are considered useful for the treatment, prevention or alleviation of pain, mild or moderate or severe pain, pain of acute, chronic or recurrent character, pain caused by migraine, postoperative pain, phantom limb pain, inflammatory pain, neuropathic pain, chronic headache, central pain, pain related to diabetic neuropathy, to post therapeutic neuralgia, or to peripheral nerve injury.

In a fourth more preferred embodiment, the pyrazole derivatives of the invention are considered useful for the treatment, prevention or alleviation of cardiac ischemia, ischemic heart disease, hypertrophic heart, cardiomyopathy or failing heart.

In a fifth more preferred embodiment, the compounds of the invention are considered useful for the treatment, prevention or alleviation of a cardiovascular disease. In a more preferred embodiment the cardiovascular disease is atherosclerosis, ischemia/reperfusion, hypertension, restenosis, arterial inflammation, myocardial ischaemia or ischaemic heart disease.

In an sixth more preferred embodiment, the compounds of the invention are considered useful for obtaining preconditioning of the heart. Preconditioning, which includes ischemic preconditioning and myocardial preconditioning, describes short periods of ischemic events before initiation of a long lasting ischemia. The compounds of the invention are believed having an effect similar to preconditioning obtained by such ischemic events. Preconditioning protects against later tissue damage resulting from the long lasting ischemic events.

In a seventh more preferred embodiment, the pyrazole derivatives of the invention are considered useful for the treatment, prevention or alleviation of schizophrenia, depression or Parkinson's disease.

In an eighth more preferred embodiment, the compounds of the invention are considered useful for the treatment, prevention or alleviation of an obstructive or inflammatory airway disease. In a more preferred embodiment the obstructive or inflammatory airway disease is respiratory failure, adult respiratory distress syndrome, asthma, nocturnal asthma, exercise induced bronchospasm, chronic obstructive pulmonary disease, giant bullae, acute bronchitis, chronic bronchitis, emphysema, reversible obstructive airway disease, bronchiectasis, bronchiolitis, cystic fibrosis, eatelectasis, pulmonary embolism, pneumonia, gastroesophageal reflux disease (GERD), lung abscess, hypersensitivity of the lung, hypersensitivity pneumonitis, eosinophilic pneumonias, allergic bronchopulmonary aspergillosis, or Goodpasture's syndrome. In an even more preferred embodiment the obstructive or inflammatory airway disease is an airway hyperreactivity, a pneumoconiosis such as aluminosis, anthracosis, asbestosis, chalicosis, ptilosis, siderosis, silicosis, tabacosis and byssinosis, a chronic obstructive pulmonary disease (COPD), bronchitis, excerbation of airways hyperreactivity or cystic fibrosis.

In its most preferred embodiment the obstructive airway disease is chronic obstructive pulmonary disease (COPD).

In a ninth more preferred embodiment the compound of the invention is used in a combination with conventional bronchodilators, in particular the beta(2)-adrenoceptor agonists. Examples of bronchodilator drugs for use according to the invention include salbutamol (Albuterol, Ventolin) and formoterol (Foradil).

In a tenth more preferred embodiment the pyrazole derivatives of the invention are considered useful for the treatment, prevention or alleviation of a sexual dysfunction, incl. male sexual dysfunction and female sexual dysfunction, and incl. male erectile dysfunction.

In an even more preferred embodiment the pyrazole derivatives of the invention may be co-administered with a phosphodiesterase inhibitor, in particular a phosphodiesterase 5 (PDE5) inhibitor, e.g. sildenafil, tadalafil, vardenafil and dipyridamole, or with an agent that potentiates endothelium-derived hyperpolarizing factor-mediated responses, in particular calcium dobesilate or similar 2,5-dihydroxybenzenesulfonate analogs.

In a most preferred embodiment the pyrazole derivatives of the invention is used in a combination therapy together with sildenafil, tadalafil, vardenafil or calcium dobesilate.

It is at present contemplated that a suitable dosage of the active pharmaceutical ingredient (API) is within the range of from about 0.1 to about 1000 mg API per day, more preferred of from about 10 to about 500 mg API per day, most preferred of from about 30 to about 100 mg API per day, dependent, however, upon the exact mode of administration, the form in which it is administered, the indication considered, the subject and in particular the body weight of the subject involved, and further the preference and experience of the physician or veterinarian in charge.

Preferred pyrazole derivatives of the invention show a biological activity in the sub-micromolar and micromolar range, i.e. of from below 1 to about 100 μM.

### Pharmaceutical Compositions

In another aspect the invention provides novel pharmaceutical compositions comprising a therapeutically effective amount of a pyrazole derivative of the invention.

While a pyrazole derivative of the invention for use in therapy may be administered in the form of the raw chemical compound, it is preferred to introduce the active ingredient, optionally in the form of a physiologically acceptable salt, in a pharmaceutical composition together with one or more adjuvants, excipients, carriers, buffers, diluents, and/or other customary pharmaceutical auxiliaries.

In a preferred embodiment, the invention provides pharmaceutical compositions comprising the pyrazole derivative of the invention together with one or more pharmaceutically acceptable carriers therefore, and, optionally, other therapeutic and/or prophylactic ingredients, know and used in the art. The carrier(s) must be “acceptable” in the sense of being compatible with the other ingredients of the formulation and not harmful to the recipient thereof.

The pharmaceutical composition of the invention may be administered by any convenient route, which suits the desired therapy. Preferred routes of administration include oral administration, in particular in tablet, in capsule, in dragé, in powder, or in liquid form, and parenteral administration, in particular cutaneous, subcutaneous, intramuscular, or intravenous injection. The pharmaceutical composition of the invention can be manufactured by any person skilled in the art, by use of standard methods and conventional techniques, appropriate to the desired formulation. When desired, compositions adapted to give sustained release of the active ingredient may be employed.

Further details on techniques for formulation and administration may be found in the latest edition of Remington's Pharmaceutical Sciences (Maack Publishing Co., Easton, Pa.).

The actual dosage depends on the nature and severity of the disease being treated, and is within the discretion of the physician, and may be varied by titration of the dosage to the particular circumstances of this invention to produce the desired therapeutic effect. However, it is presently contemplated that pharmaceutical compositions containing of from about 0.1 to about 500 mg of active ingredient per individual dose, preferably of from about 1 to about 100 mg, most preferred of from about 1 to about 10 mg, are suitable for therapeutic treatments.

The active ingredient may be administered in one or several doses per day. A satisfactory result can, in certain instances, be obtained at a dosage as low as 0.1 μg/kg i.v. and 1 μg/kg p.o. The upper limit of the dosage range is presently considered to be about 10 mg/kg i.v. and 100 mg/kg p.o. Preferred ranges are from about 0.1 μg/kg to about 10 mg/kg/day i.v., and from about 1 μg/kg to about 100 mg/kg/day p.o.

### Methods of Therapy

In another aspect the invention provides a method of treatment, prevention or alleviation of a disease, disorder or condition of a living animal body, including a human, which disorder, disease or condition is responsive to activation of a potassium channel, which method comprises the step of administering to such a living animal body in need thereof, a therapeutically effective amount a compound capable of activating the potassium channel, or a pharmaceutically-acceptable addition salt thereof.

The preferred medical indications contemplated according to the invention are those stated above.

It is at present contemplated that a suitable dosage of the active pharmaceutical ingredient (API) is within the range of from about 0.1 to about 1000 mg API per day, more preferred of from about 1 to about 500 mg API per day, most preferred of from about 1 to about 100 mg API per day, dependent, however, upon the exact mode of administration, the form in which it is administered, the indication considered, the subject and in particular the body weight of the subject involved, and further the preference and experience of the physician or veterinarian in charge.

### BRIEF DESCRIPTION OF THE DRAWINGS

The present invention is further illustrated by reference to the accompanying drawing, in which FIGS. 1A and 1B show the effect of Compound 9 (i.e. 5-{2-[1-(4-Bromo-phenyl)-3-(4-chloro-phenyl)-1H-pyrazol-4-yl]-ethyl}-1H-tetrazole) on the voltage dependence of BK_{ca} channels expressed in Xenopus oocytes:

FIG. 1A shows conductance (μS) vs. membrane potential (mV) in the absence (Control) of Compound 9 and in the presence of 0.01 to 31.6 μM of Compound 9;

FIG. 1B shows the concentration-response relationship for the left-shift of the BK_{Ca}-activation curve induced by Compound 9; i.e. ΔV (mV) vs. log [c] (M). The calculated EC₅₀-value is 1.4 μM and the maximal left-shift for the BK-activation curve is −96 mV.

### EXAMPLES

The invention is further illustrated with reference to the following examples, which are not intended to be in any way limiting to the scope of the invention as claimed.

### Example 1

### Preparatory Example

Abbreviations Used Herein:

AcOEt: ethyl acetate

CFM: chloroform

DCM: dichloromethane

DMF: N,N-dimethylformamide

DMAP: 4-dimethylaminopyridine

EDC.HCl: 1-ethyl-3-(3-dimethylaminopropyl)carbodiimide hydrochloride

EtOH: ethanol 99%

Hex: hexane

MeOH: methanol

MgSO4: magnesium sulphate

Py: pyridine

THF: tetrahydrofuran

TOL: toluene

### (E)-3-(1,3-diphenyl-1H-pyrazol-4-yl)-acrylic Acids and Bioisosteric Derivatives of the Carboxylic Moiety (a) (Scheme 1)

The synthesis of (E)-3-(1,3-diphenyl-1H-pyrazol-4-yl)acrylic acids usually begins with the condensation between commercially-available ketones and hydrazines, to afford the correspondent hydrazones. The Vilsmeier-Haack reaction on these latters, followed by aqueous hydrolysis of the Vilsmeier adduct, provides the desired cyclised aldehyde, as widely reported in the literature (e.g. by Rathelot, Pascal et al.; European Journal of Medicinal Chemistry 2002 37 (8) 671-679). Condensation of the resulting aldehyde with malonic acid provides the correspondent (E)-3-(1,3-diphenyl-1H-pyrazol-4-yl)-acrylic acids (see e.g. Bernard M et al.; Pharmazie 1986 41 (8) 560-562). As an example for this general experimental procedure, the synthesis of compound I is described herein and outlined in Scheme 1.

As a further example of this class of compounds, the chemical-physical properties for compound 3 are reported.

Preparation of derivatives of the (E)-3-(1,3-diphenyl-1H-pyrazol-4-yl)-acrylic acids, such as N-sulfonyl-carboxamide and N-cyano-carboxamide derivatives involves the use of the same (E)-3-(1,3-diphenyl-1H-pyrazol-4-yl)-acrylic acids as starting points and conversion to the correspondent derivatives by well-know synthetic procedures to those skilled in the art. As an example of these experimental procedures, the synthesis of the N-cyano-carboxamide 2 is reported and outlined in Scheme 1.

The N-sulfonyl-carboxamide 6, whose chemical physical properties have been reported below, is obtained in a similar manner, from the correspondent acid 3 upon treatment with methanesulfonamide.

Preparation of more biososteric derivatives of (E)-3-(1,3-diphenyl-1H-pyrazol-4-yl)-acrylic acids is also reported herein, such as 5-[(E)-2-(1,3-diphenyl-1H-pyrazol-4-yl)-vinyl]-1H-tetrazoles or 3-[(E)-2-(1,3-diphenyl-1H-pyrazol-4-yl)-vinyl]-2H-[1,2,4]-oxadiazol-5-ones. Both derivatives can been synthesised from the suitable α,β-unsaturated nitriles, prepared by the Horner-Hemmons reaction between the suitable 1,3-diphenyl-1H-pyrazole-4-carbaldehyde and diethylcyanomethylphosphonate (see e.g. Majetich G et al.; Journal of Organic Chemistry 1986 51 (10) 1745-53). The ciano derivatives are finally converted to the correspondent tetrazole derivatives, upon treatment with azidotributyltin or to the correspondent oxadiazolone derivatives (e.g. as described by Valgeirsson et al. in Journal of Medicinal Chemistry 2004 47 (27) 6948-6957). As an example, the synthesis of the tetrazole derivative 4 and the chemical physical properties of 5 are described herein and outlined in Scheme 1.

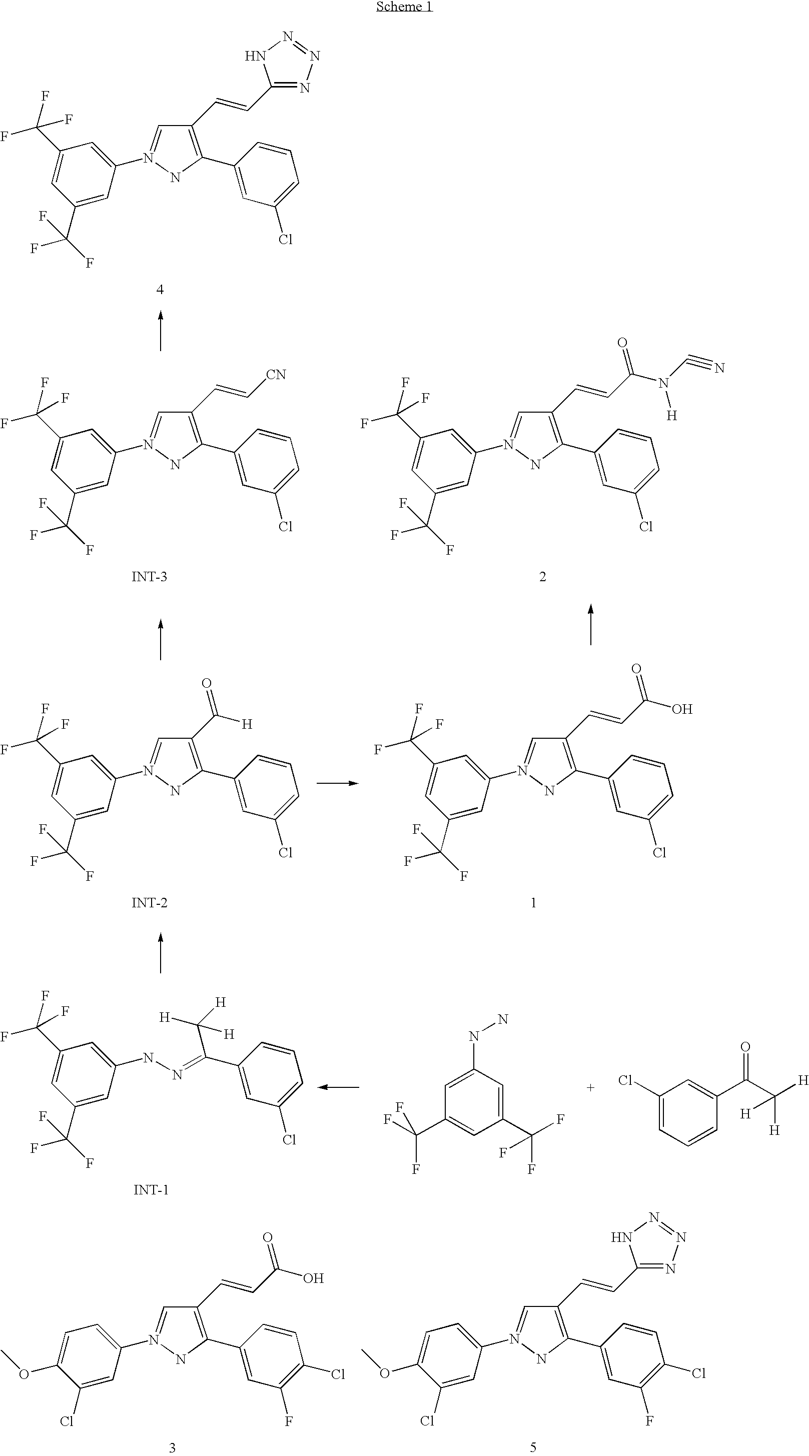

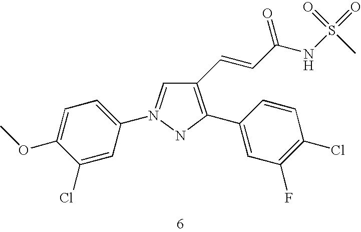

### 3-(1,3-diphenyl-1H-pyrazol-4-yl)-propionic Acids and Biososteric Derivatives of the Carboxylic Acid Moiety (b) (Scheme 2)

Preparation of derivatives of 3-(1,3-diphenyl-1H-pyrazol-4-yl)-propionic acids, such as N-sulfonyl-carboxamides and N-cyano-carboxamides, involves the 3-(1,3-diphenyl-1H-pyrazol-4-yl)-propionic acids as starting reagents. These latter can be either commercial or synthesised by the suitably substituted 1,3-diphenyl 1H-pyrazole-4-carbaldehydes, prepared as described above or in Synthetic communications 25 (19), 3067-3074, 1995). Alternatively, the 3-(1,3-diphenyl-1H-pyrazol-4-yl)-propionic acids can be obtained by the reduction of the above described (E)-3-(1,3-diphenyl-1H-pyrazol-4-yl)-acrylic acids (following the experimental procedure described in Bernard M et al.; Pharmazie 1986 41 (8) 560-562). As an example of 3-(1,3-diphenyl-1H-pyrazol-4-yl)-propionic acids and bioisosteric derivatives, the synthesis of compounds 7, 8, 9, 11 and the chemical physical properties of 10 are described herein and outlined in Scheme 2.

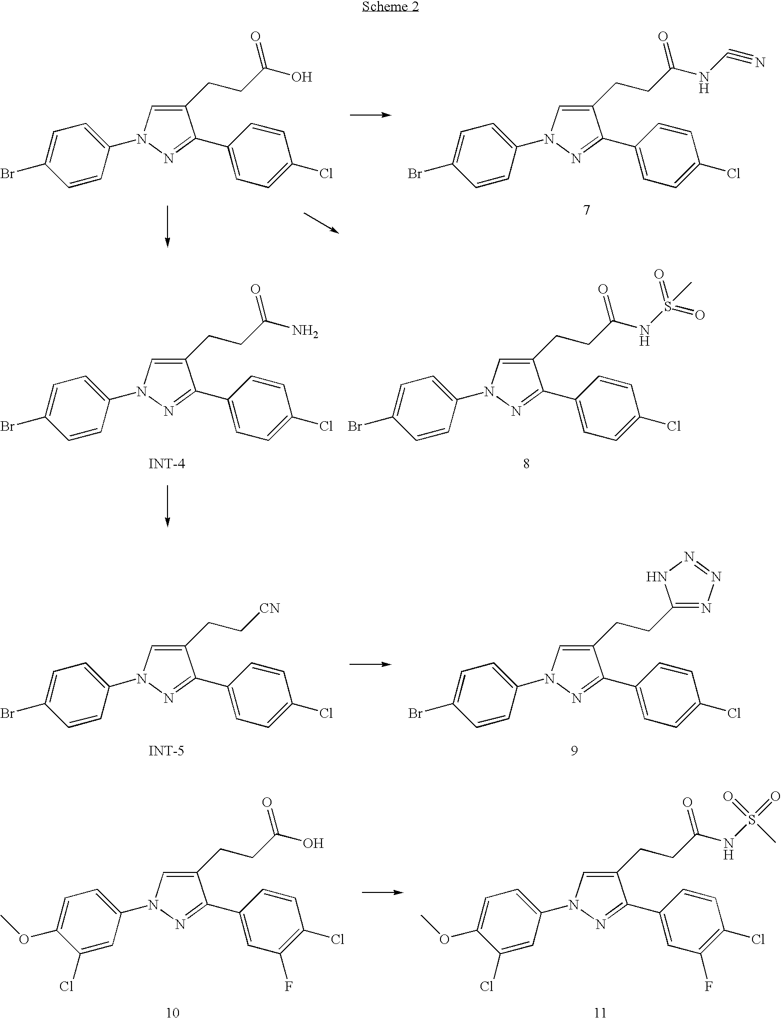

### 2-Cyano-3-(1,3-diphenyl-1H-pyrazol-4-yl)-acrylic Acids and Bioisosteric Derivatives of the Carboxylic Acid Moiety (c) (Scheme 3)

Preparation of 2-cyano-3-(1,3-diphenyl-1H-pyrazol-4-yl)-acrylic acids were prepared by straightforward Knoevenagel condensation of the suitable 1,3-diphenyl 1H-pyrazole-4-carbaldehydes with cyanoacetic acid tert-butyl ester, as described by Gazit et. Al. in Journal of Medicial Chemistry 32, 2344-2352, 1989 and the resulting carboxylic acids were then further derivatized to bioisosteric analogues. As an example, the synthesis and/or the chemical physical properties of the compounds 12-16 are described herein and outlined in Scheme 3.

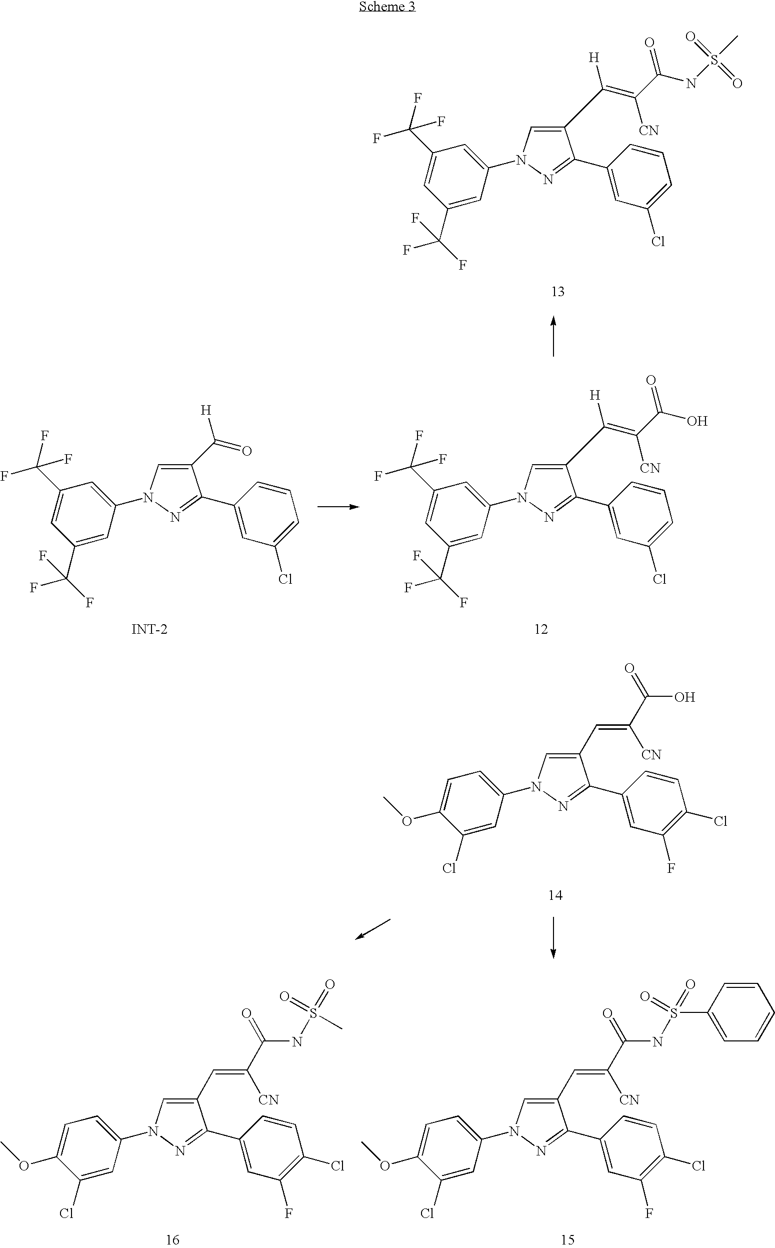

### N-(3,5-Bis-trifluoromethyl-phenyl)-N′-[1-(3-chloro-phenyl)-ethylidene]-hydrazine (INT-1)

To a stirred solution of commercial 3,5-bis(trifluoromethyl)phenyl) hydrazine (2.00 g, 1 eq) in EtOH (20 ml), 3-chloroacetophenone (1.27 g, 1 eq) is slowly added. The reaction mixture is refluxed for 4 hours and then poured into water (70 ml): the new mixture is extracted with AcOEt and the organic phase is washed with water, dried over MgSO₄ and evaporated to dryness, to afford the title compound (3.11 g, 100% yield), which is used as such for the next step.

### 5-(3-Chloro-phenyl)-2-[3-(1,1-difluoro-ethyl)-5-trifluoromethyl-phenyl]-1H-pyrazole-4-carbaldehyde (INT-2)

Phosphorus oxychloride (2.4 ml, 3 eq) is added to 18 ml of DMF at 0° C. and stirred for 30 minutes. To this mixture, INT-1 (3.2 g, 1 eq) is slowly added and the new reaction mixture is stirred at room temperature for 5 hours and then quenched into water (120 ml) and stirred for an additional 5 hours. The resulting solid is filtered and dried, to give the title compound as white solid (2.00 g, 57% yield), which is used as such for the next step.

### (E)-3-[2-(3,5-Bis-trifluoromethyl-phenyl)-5-(3-chloro-phenyl)-1H-pyrazol-4-yl]-acrylonitrile (INT-3)

Diethyl (cyanomethyl) phosphonate (0.507 g, 1.2 eq) is added to a stirred suspension of sodium hydride (0.115, 1.2 eq) in THF (5 ml) at 0° C. and stirring is continued for 15 minutes at 0° C. To this latter mixture (cooled to −78° C.), a solution of INT-2 (1.00 g, 1 eq) in THF (10 ml) is added drop wise. The reaction mixture is allowed to reach room temperature over a period of 1 hour and it is then quenched into water (60 ml), extracted with DCM (60 ml×3), washed with 1.5 N HCl and water, dried over MgSO₄ and evaporated to dryness. The resulting solid residue is purified by flash column chromatography using silica gel (230-400 mesh) and eluting with 4% AcOEt in Hex, in order to afford the title compound as an off-white solid (0.50 g, yield ˜50%).

### 3-[1-(4-Bromo-phenyl)-3-(4-chloro-phenyl)-1H-pyrazol-4-yl]-propionamide (INT-4)

A mixture of the commercial 3-[1-(4-bromo-phenyl)-3-(4-chloro-phenyl)-1H-pyrazol-4-yl]-propionic acid (1.00 g) and oxalyl chloride (10 ml) is heated (50° C.) for 2 hours and then evaporated to dryness. The resulting yellow solid is dissolved in THF (13 ml), the solution is cooled to −30° C. and ammonia gas is bubbled in for 30 min. The reaction mixture is allowed to attain reach temperature spontaneously and evaporated to dryness. The new solid is washed several times with water, dried (0.70 g, 70% yield) and used as such for the next step.

### 3-[1-(4-Bromo-phenyl)-3-(4-chloro-phenyl)-1H-pyrazol-4-yl]-propionitrile (INT-5)

A mixture of 0.5 g of INT-4, toluene (5 ml) and phosphorus oxychloride (0.5 ml) is refluxed for 6 hours and evaporated to dryness afterwards. The resulting solid (0.47 g) is purified by flash chromatography using 0-0.2% of MeOH in CFM as eluent, to give the title compound as off-white solid (0.425 g, 89% yield). IR (cm⁻¹): 2241.

### (E)-3-[1-(3,5-Bis-trifluoromethyl-phenyl)-3-(3-chloro-phenyl)-1H-pyrazol-4-yl]-acrylic acid (1)

To a stirred solution of INT-2 (0.300 g, 1 eq) in Py (5 ml), piperidine (0.1 ml) and malonic acid (0.149 g, 2 eq) are added and the mixture is refluxed for 12 hours. The reaction mixture is poured onto ice (30 ml) and stirred for 2 hours. The resulting solid is filtered and dried (0.260 g) and purified by crystallisation from a mixture of DCM and Hex, to afford the title compound as an off-white solid (0.095 g, 28% yield). Mp. 217.2-220.6° C.

### (E)-3-[1-(3,5-Bis-trifluoromethyl-phenyl)-3-(3-chloro-phenyl)-1H-pyrazol-4-yl]-N-cyanoacrylamide (2)

Compound I (1.00 g, 1 eq) is dissolved in an excess of oxalyl chloride (20 ml) and refluxed for 4 hours. The reaction mixture is evaporated to dryness and the resulting solid residue is taken up in dry THF (5 ml) at 0° C. To this solution, an ice-cooled suspension of sodium hydride (0.174, 2 eq) and cyanamide (0.137 g, 1 eq) in THF (10 ml) is added and stirring is continued at room temperature overnight. The new reaction mixture is poured into an aqueous saturated solution of sodium bicarbonate (50 ml) and extracted with AcOEt (2×50 ml). The organic phase is dried over MgSO₄ and evaporated to dryness to get a brownish semisolid (1.10 g). This crude residue is purified by flash column chromatography using silica gel (230-400 mesh) and eluting with 4% CFM in MeOH, to afford the title compound as a yellow solid (0.080 g, yield 7%).

### (E)-3-[3-(4-Chloro-3-fluoro-phenyl)-1-(3-chloro-4-methoxy-phenyl)-1H-pyrazol-4-yl]-acrylic acid (3)

M.p. 240.2-241.3° C. LC-ESI-HRMS of [M+H]+ shows 407.0356 Da. Calc. 407.036552 Da, dev. −2.3 ppm.

### 5-{(E)-2-[1-(3,5-Bis-trifluoromethyl-phenyl)-3-(3-chloro-phenyl)-1H-pyrazol-4-yl]-vinyl}-1H-tetrazole (4)

To a suspension of INT-3 (0.350 g, 1 eq) in TOL (5 ml), azidotributyltin (0.395 g, 1.5 eq) is added and the mixture is refluxed for 12 hours. The reaction mixture is then diluted with AcOEt (60 ml), washed with water, dried over MgSO₄ and evaporated to dryness, to provide a brownish gummy material (0.510 g). This crude residue is purified by flash column chromatography using silica gel (230-400 mesh) and eluting with 2% MeOH in CFM, to afford the title compound as an off-white solid (0.13 g, 34% yield). Mp. 227.8-229.3° C.

### 5-{(E)-2-[3-(4-Chloro-3-fluorophenyl)-1-(3-chloro-4-methoxy-phenyl)-1H-pyrazol-4-yl]-vinyl}-1H-tetrazole (5)

M.p. 128.4-130.0° C. LC-ESI-HRMS of [M+H]+ shows 431.0593 Da. Calc. 431.059018 Da, dev. 0.7 ppm.

### N-{(E)-3-[3-(4-Chloro-3-fluoro-phenyl)-1-(3-chloro-4-methoxy-phenyl)-1H-pyrazol-4-yl]-acryloyl}-methanesulfonamide (6)

M.p. 269.3-270.5° C. LC-ESI-HRMS of [M+H]+ shows 484.0318 Da. Calc. 484.030087 Da, dev. 3.5 ppm.

### 3-[1-(4-Bromo-phenyl)-3-(4-chloro-phenyl)-1H-pyrazol-4-yl]-N-cyanopropionamide (7)

To a stirred and ice-cooled solution of 3-[1-(4-bromo-phenyl)-3-(4-chloro-phenyl)-1H-pyrazol-4-yl]-propionyl chloride (0.656 g, 1 eq) (prepared by the correspondent 3-[1-(4-bromo-phenyl)-3-(4-chloro-phenyl)-1H-pyrazol-4-yl]-propionic acid upon treatment with oxalyl chloride) in dry THF (20 ml), sodium hydrogencyanamide (0.297 g, 3 eq) is added and stirring is continued overnight at room temperature. The reaction is quenched by addition of water (30 ml) and extraction with AcOEt follows. The organic phase is washed with water, dried over MgSO₄ and evaporated to dryness. The resulting crude solid residue is purified by flash chromatography using silica gel (230-400 mesh) and eluting with 0-10% MeOH in DCM, to afford the title compound as white powder (0.224 g, ˜33% yield). LC-ESI-HRMS of [M+H]+ shows 429,014 Da. Calc. 429,011777 Da, dev. 5.2 ppm.

### N-{3-[1-(4-Bromo-phenyl)-3-(4-chloro-phenyl)-1H-pyrazol-4-yl]-propionyl}-methanesulfonamide (8)

To a mixture of 3-[1-(4-bromo-phenyl)-3-(4-chloro-phenyl)-1H-pyrazol-4-yl]-propionic acid (0.620 g, 1 eq) in DCM (25 ml), EDC.HCl (0.586 g, 2 eq) and DMAP (0.560 g, 3 eq) are added. The resulting brown solution is stirred for 10 minutes and methanesulfonamide (0.145 g, 1 eq) is then added. The reaction mixture is stirred at room temperature overnight, diluted with DCM (20 ml), washed with 1.5 N HCl (2×40 ml), water (25 ml) and finally dried over MgSO₄ and evaporated to dryness, to give a yellowish solid (0.702 g). This crude material is purified by flash chromatography using silica gel (230-400 mesh) and eluting with 0-10% MeOH in DCM, to afford the title compound as white powder (0.403 g, ˜55% yield). LC-ESI-HRMS of [M+H]+ shows 481,9937 Da. Calc. 481,994079 Da, dev. −0.8 ppm.

### 5-{2-[1-(4-Bromo-phenyl)-3-(4-chloro-phenyl)-1H-pyrazol-4-yl]-ethyl}-1H-tetrazole (9)

A mixture of INT-5 (0.275 g, 1 eq), sodium azide (0.462 g, 10 eq), ammonium chloride (0.388 g, 10 eq) and DMF (2 ml) is heated (microwave oven, 120° C.) for 3 hours. The reaction mixture is dilute with DCM (15 ml) and washed with HCl 1.5 N (20 ml). The organic phase, dried over MgSO₄ and evaporated to dryness, provided a solid residue (0.40 g) which is purified by flash chromatography by eluting with 0-99% AcOEt in Hex (0.08, yield ˜40%). M.p. 164.2-165.9° C. LC-ESI-HRMS of [M+H]+ shows 429.0226 Da. Calc. 429.02301 Da, dev. −1 ppm.

### 3-[3-(4-Chloro-3-fluoro-phenyl)-1-(3-chloro-4-methoxy-phenyl)-1H-pyrazol-4-yl]-propionic acid (10)

LC-ESI-HRMS of [M+H]+ shows 409.0517 Da. Calc. 409.052202 Da, dev. −1.2 ppm.

### N-{3-[3-(4-Chloro-3-fluoro phenyl)-1-(3-chloro-4-methoxy-phenyl)-1H-pyrazol-4-yl]-propionyl}-methanesulfonamide (11)

To a mixture of 10 (0.147 g, 1 eq) in DCM (5 ml), EDC.HCl (0.1377 g, 2 eq) and DMAP (0.1317 g, 3 eq) are added. The resulting brown solution is stirred for 10 minutes and methanesulfonamide (0.0342 g, 1 eq) is then added. The reaction mixture is stirred at room temperature overnight, diluted with DCM (15 ml), washed with 1.5 N HCl (2×20 ml), water (20 ml) and finally dried over MgSO₄ and evaporated to dryness, to give a white solid (0.090 g). This material (99.8% pure, yield 51%) is not purified further. M.p. 211.2-212.5° C. LC-ESI-HRMS of [M+H]+ shows 486.0443 Da. Calc. 486.045737 Da, dev. −3 ppm.

### (E)-3-[1-(3,5-Bis-trifluoromethyl-phenyl)-3-(3-chloro-phenyl)-1H-pyrazol-4-yl]-2-cyano-acrylic acid (12)

H1-NMR (DMSO-d₆): δ 13.99 (bs, 1H), 9.55 (s, 1H), 8.69 (s, 2H), 8.21 (s, 1H), 8.05 (s, 1H), 7.80 (s, 1H), 7.63-7.61 (m, 3H).

### N-{(E)-3-[1-(3,5-Bis-trifluoromethyl-phenyl)-3-(3-chloro-phenyl)-1H-pyrazol-4-yl]-2-cyano-acryloyl}-methanesulfonamide (13)

To a mixture of 12 (0.5 g, 1 eq) in DCM (7 ml), EDC.HCl (0.3946 g, 2 eq) and DMAP (0.377 g, 3 eq) are added. The resulting brown solution is stirred for 10 minutes and methanesulfonamide (0.0979 g, 1 eq) is then added. The reaction mixture is stirred at room temperature overnight, diluted with DCM (20 ml), washed with 1.5 N HCl (2×30 ml), water (25 ml) and finally dried over MgSO₄ and evaporated to dryness, to give a yellowish solid (0.450 g). This crude material is purified by preparative HPLC, to afford the title compound as white powder (0.83 g, ˜15% yield). M.p. 273.2-274.8° C.

### (E)-3-[3-(4-Chloro-3-fluoro-phenyl)-1-(3-chloro-4-methoxy-phenyl)-1H-pyrazol-4-yl]-2-cyano-acrylic acid (14)

M.p. 282.8-284.2° C. LC-ESI-HRMS of [M+H]+ shows 432.0298 Da. Calc. 432.031801 Da, dev. −4.6 ppm.

### N-{(E)-3-[3-(4-Chloro-3-fluoro-phenyl)-1-(3-chloro-4-methoxy-phenyl)-1H-pyrazol-4-yl]-2-cyano-acryloyl}-benzenesulfonamide (15)

To a mixture of 14 (0.900 g, 1 eq) in DCM (30 ml), EDC.HCl (0.7983 g, 2 eq) and DMAP (0.3053 g, 1.2 eq) are added. The resulting brown solution is stirred for 10 minutes and benzenesulfonamide (0.3273 g, 1.2 eq) is then added. The reaction mixture is stirred at room temperature overnight, diluted with DCM (80 ml), washed with 1.5 N HCl (2×110 ml), water (100 ml) and finally dried over MgSO₄ and evaporated to dryness, to give a yellowish solid (0.850 g). This crude material is purified by flash chromatography using with 3% MeOH in CFM as eluent, to afford the title compound as white powder (0.168 g, ˜14% yield). M.p. 263.2-264.3° C.

### N-{(E)-3-[3-(4-Chloro-3-fluorophenyl)-1-(3-chloro-4-methoxy-phenyl)-1H-pyrazol-4-yl]-2-cyano-acryloyl}-methanesulfonamide (16)

To a mixture of 14 (1.00 g, 1 eq) in DCM (25 ml), EDC.HCl (0.887 g, 2 eq) and DMAP (0.3392 g, 1.2 eq) are added. The resulting brown solution is stirred for 10 minutes and methanesulfonamide (0.2201 g, 1.2 eq) is then added. The reaction mixture is stirred at room temperature overnight, diluted with DCM (30 ml), washed with 1.5 N HCl (2×50 ml), water (50 ml) and finally dried over MgSO₄ and evaporated to dryness, to give a yellowish solid (0.750 g). This crude material is purified by flash chromatography using with 3% MeOH in CFM as eluent and recrystallised from a mixture of DCM and Hex, to afford the title compound as white powder (0.149 g, ˜13% yield). M.p. 280.2-281.5° C. LC-ESI-HRMS of [M−H]− shows 507.0107 Da. Calc. 507.009686 Da, dev. 2 ppm.

### 3-[1-(3,5-Bis-trifluoromethyl-phenyl)-3-(3-chloro-phenyl)-1H-pyrazol-4-ylmethyl]-4H-[1,2,4]oxadiazol-5-one (17)

M.p. 200-201° C. LC-ESI-HRMS of [M−H]− shows 487.0381 Da. Calc. 487.039647 Da, dev. −3.2 ppm.

### N-{2-[3-(4-Chloro-3-fluoro phenyl)-1-(3-chloro-4-methoxy-phenyl)-1H-pyrazol-4-yl]-acetyl}-methanesulfonamide (18)

M.p. 216.8-218.2° C. LC-ESI-HRMS of [M+H]+ shows 472.0296 Da. Calc. 472.030087 Da, dev. −1 ppm.

### 5-[3-(4-Chloro-3-fluoro phenyl)-1-(3-chloro-4-methoxy-phenyl)-1H-pyrazol-4-ylmethyl]-H-tetrazole (19)

LC-ESI-HRMS of [M+H]+ shows 419.0609 Da. Calc. 419.059018 Da, dev. 4.5 ppm.

### Example 2

### Biological Activity

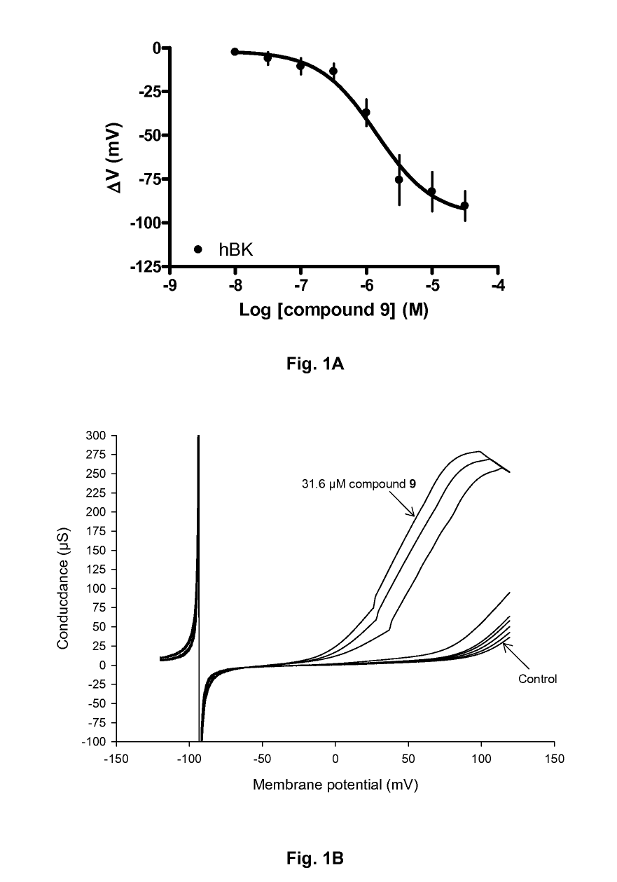

In this example the BK channel opening activity of Compound 9 (FIGS. 1A and 1B) is determined using BK channels heterologously expressed in Xenopus laevis oocytes.

The electrical current through the BK channel was measured using conventional two-electrode voltage clamp. BK currents were activated by repeating ramp protocols. In brief, the membrane potential was continuously changed from −120 mV to +120 mV within a 2 s period. The threshold for BK activation is approximately +30 mV under control conditions. Compounds were applied for 100 s during which the ramp protocol was repeated 10 times with 10 s intervals. In between the ramp protocols the membrane potential was clamped at −80 mV. The first three compound applications were control blanks where the current level is allowed to stabilize. During the subsequent 8 applications increasing concentrations (0.01-31.6 μM) of Compound 9 was applied and a marked increase in the current level at depolarizing potentials was observed.

In order to evaluate the ability of the compounds to shift the BK activation curve towards lower membrane potentials, the BK current was transformed into conductance by using Ohm's law g=1/(E_{memb}−Eᵣₑᵥ), where g is the conductance, I is the current, E_{memb} is the membrane potential and Eᵣₑᵥ is the reversal potential. The extracellular solution for these experiments contained 2.5 mM K+ and the intracellular K+ concentration of an oocyte was estimated to be 100 mM. Under those conditions, Nernst equation predicts a reversal potential of Eᵣₑᵥ=−93.2 mV. The control conductance level at a membrane potential of +100 mV was calculated, and the compound effect was evaluated as the potential difference, ΔV, to the membrane potential at which the same conductance level was obtained in the presence of compound.

The concentration response curve for this potential difference was fitted to the sigmoidal logistic equation: ΔV=ΔVₘₐₓ/(1+(EC₅₀/[compound])ⁿ), where ΔVₘₐₓ represents the maximal left shift of the BK activation curve, EC₅₀ is the concentration causing a half maximal response, and n is the slope coefficient.

The calculated EC₅₀ and ΔVmax values for Compound 9 were 1.4 μM and −96 mV, respectively.

1-10. (canceled)

11. A pyrazole derivative of Formula I 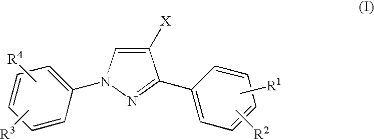
an isomer thereof or a mixture of its isomers, or an N-oxide thereof, or a pharmaceutically acceptable salt thereof, wherein
X represents
a tetrazolyl-alkyl group, an oxadiazolonyl-alkyl group, an [(N-alkyl-sulfonyl)carbamoyl]-alkyl group, 2-cyano-acrylic acid, 2-cyano-acryloyl-alkylsulfonamide or 2-cyano-acryloyl-phenylsulfonamide;
or a group of formula CH═CH—W or CH₂—CH₂—W, wherein W represents a tetrazolyl group, N-alkylsulfonylcarboxamide, carboxy, N-cyanocarboxamide or 5-oxo-4,5-dihydro-[1,2,4]oxadiazol-3-yl;
R¹ and R², independently of each other, represent hydrogen, halo, hydroxy or phenyl, which phenyl may optionally be optionally substituted one or more times with halo; and
R³ and R⁴, independently of each other, represent hydrogen, halo, trifluoromethyl, hydroxy, alkoxy or phenyl.

12. The pyrazole derivative of claim 11, an isomer thereof or a mixture of its isomers, or an N-oxide thereof, or a pharmaceutically acceptable salt thereof, wherein X represents a tetrazolyl-alkyl group, an oxadiazolonyl-alkyl group, an [(N-alkyl-sulfonyl)carbamoyl]-alkyl group, 2-cyano-acrylic acid, 2-cyano-acryloyl-alkylsulfonamide or 2-cyano-acryloyl-phenylsulfonamide.

13. The pyrazole derivative of claim 11, an isomer thereof or a mixture of its isomers, or an N-oxide thereof, or a pharmaceutically acceptable salt thereof, wherein
X represents a group of formula CH═CH—W or CH₂—CH₂—W, and wherein
W represents a tetrazolyl group, N-alkylsulfonylcarboxamide, carboxy, N-cyanocarboxamide or 5-oxo-4,5-dihydro-[1,2,4]oxadiazol-3-yl.

14. The pyrazole derivative of any one of claims 11-13, an isomer thereof or a mixture of its isomers, or an N-oxide thereof, or a pharmaceutically acceptable salt thereof, wherein R¹ and R², independently of each other, represent hydrogen, halo, hydroxy or phenyl, which phenyl may optionally be optionally substituted one or more times with halo.

15. The pyrazole derivative of claim 11, an isomer thereof or a mixture of its isomers, or an N-oxide thereof, or a pharmaceutically acceptable salt thereof, wherein R³ and R⁴, independently of each other, represent hydrogen, halo, trifluoromethyl, hydroxy, alkoxy or phenyl.

16. The pyrazole derivative of claim 11, which is
(E)-3-[1-(3,5-Bis-trifluoromethyl-phenyl)-3-(3-chloro-phenyl)-1H-pyrazol-4-yl]-acrylic acid;
(E)-3-[1-(3,5-Bis-trifluoromethyl-phenyl)-3-(3-chloro-phenyl)-1H-pyrazol-4-yl]-N-cyanoacrylamide;
(E)-3-[3-(4-Chloro-3-fluoro-phenyl)-1-(3-chloro-4-methoxy-phenyl)-1H-pyrazol-4-yl]-acrylic acid;
5-{(E)-2-[1-(3,5-Bis-trifluoromethyl-phenyl)-3-(3-chloro-phenyl)-1H-pyrazol-4-yl]-vinyl}-1H-tetrazole;
5-{(E)-2-[3-(4-Chloro-3-fluoro-phenyl)-1-(3-chloro-4-methoxy-phenyl)-1H-pyrazol-4-yl]-vinyl}-1H-tetrazole;
N-{(E)-3-[3-(4-Chloro-3-fluoro-phenyl)-1-(3-chloro-4-methoxy-phenyl)-1H-pyrazol-4-yl]-acryloyl}-methanesulfonamide;
3-[1-(4-Bromo-phenyl)-3-(4-chloro-phenyl)-1H-pyrazol-4-yl]-N-cyanopropionamide;
N-{3-[1-(4-Bromo-phenyl)-3-(4-chloro-phenyl)-1H-pyrazol-4-yl]-propionyl}-methanesulfonamide;
5-{2-[1-(4-Bromo-phenyl)-3-(4-chloro-phenyl)-1H-pyrazol-4-yl]-ethyl}-1H-tetrazole;
3-[3-(4-Chloro-3-fluoro-phenyl)-1-(3-chloro-4-methoxy-phenyl)-1H-pyrazol-4-yl]-propionic acid;
N-{3-[3-(4-Chloro-3-fluoro-phenyl)-1-(3-chloro-4-methoxy-phenyl)-1H-pyrazol-4-yl]-propionyl}-methanesulfonamide;
(E)-3-[1-(3,5-Bis-trifluoromethyl-phenyl)-3-(3-chloro-phenyl)-1H-pyrazol-4-yl]-2-cyano-acrylic acid;
N-{(E)-3-[1-(3,5-Bis-trifluoromethyl-phenyl)-3-(3-chloro-phenyl)-1H-pyrazol-4-yl]-2-cyano-acryloyl}-methanesulfonamide;
(E)-3-[3-(4-Chloro-3-fluoro-phenyl)-1-(3-chloro-4-methoxy-phenyl)-1H-pyrazol-4-yl]-2-cyano-acrylic acid;
N-{(E)-3-[3-(4-Chloro-3-fluoro-phenyl)-1-(3-chloro-4-methoxy-phenyl)-1H-pyrazol-4-yl]-2-cyano-acryloyl}-benzenesulfonamide;
N-{(E)-3-[3-(4-Chloro-3-fluoro-phenyl)-1-(3-chloro-4-methoxy-phenyl)-1H-pyrazol-4-yl]-2-cyano-acryloyl}-methanesulfonamide;
3-[1-(3,5-Bis-trifluoromethyl-phenyl)-3-(3-chloro-phenyl)-1H-pyrazol-4-ylmethyl]-4H-[1,2,4]oxadiazol-5-one;
N-{2-[3-(4-Chloro-3-fluoro-phenyl)-1-(3-chloro-4-methoxy-phenyl)-1H-pyrazol-4-yl]-acetyl}-methanesulfonamide; or
5-[3-(4-Chloro-3-fluoro-phenyl)-1-(3-chloro-4-methoxy-phenyl)-1H-pyrazol-4-ylmethyl]-1H-tetrazole;
or an isomer thereof or a mixture of its isomers, or an N-oxide thereof, or a pharmaceutically acceptable salt thereof.

17. A pharmaceutical composition comprising a therapeutically effective amount of the pyrazole derivative of claim 11, an isomer thereof or a mixture of its isomers, or an N-oxide thereof, or a pharmaceutically-acceptable addition salt thereof, or a prodrug thereof, together with one or more adjuvants, excipients, carriers and/or diluents.

18. A method of treatment, prevention or alleviation of a disease or a disorder or a condition of a living animal body, including a human, which disorder, disease or condition is responsive to modulation of potassium channels, which method comprises the step of administering to such a living animal body in need thereof, a therapeutically effective amount of the pyrazole derivative according to claim 11, an isomer thereof or a mixture of its isomers, or an N-oxide thereof, or a pharmaceutically acceptable salt thereof.

19. The method according to claim 18, wherein the disease, disorder or condition is a respiratory disease, epilepsy, convulsions, seizures, absence seizures, vascular spasms, coronary artery spasms, motor neuron diseases, myokymia, renal disorders, polycystic kidney disease, bladder hyperexcitability, bladder spasms, urinogenital disorders, urinary incontinence, bladder outflow obstruction, erectile dysfunction, gastrointestinal dysfunction, gastrointestinal hypomotility disorders, gastrointestinal motility insufficiency, postoperative ileus, constipation, gastroesophageal reflux disorder, secretory diarrhoea, an obstructive or inflammatory airway disease, ischaemia, cerebral ischaemia, ischaemic heart disease, angina pectoris, coronary heart disease, ataxia, traumatic brain injury, stroke, Parkinson's disease, bipolar disorder, psychosis, schizophrenia, autism, anxiety, mood disorders, depression, manic depression, psychotic disorders, dementia, learning deficiencies, age related memory loss, memory and attention deficits, Alzheimer's disease, amyotrophic lateral sclerosis (ALS), dysmenorrhea, narcolepsy, sleeping disorders, sleep apnea, Reynaud's disease, intermittent claudication, Sjogren's syndrome, xerostomia, cardiovascular disorders, hypertension, myotonic dystrophy, myotonic muscle dystrophia, spasticity, xerostomi, diabetes Type II, hyperinsulinemia, premature labour, cancer, brain tumors, inflammatory bowel disease, irritable bowel syndrome, colitis, colitis Crohn, immune suppression, hearing loss, migraine, pain, neuropathic pain, inflammatory pain, trigeminal neuralgia, vision loss, rhinorrhoea, ocular hypertension (glaucoma) or baldness.
